(19) United States
(12) Patent Application Publication
(10) Pub. No.: US 2014/0080787 A1
(43) Pub. Date: Mar. 20, 2014

(54) Method for the Treatment of Neuropathies Associated with Charcot-Marie-Tooth 1A (CMT1A) Disease

(71) Applicants: National Institutes of Health, US Dept of Health and Human Services, Bethesda, MD (US); Charcot-Marie-Tooth Association, Chester, PA (US)

(72) Inventors: James Inglese, Bethesda, MD (US); John Peter Svaren, Madison, WI (US); Sung-Wook Jang, Germantown, MD (US); Mark Albert Scheideler, Silver Spring, MD (US); Lars Jacob Stray Knutsen, West Chester, PA (US)

(21) Appl. No.: 14/026,667

(22) Filed: Sep. 13, 2013

Related U.S. Application Data
(60) Provisional application No. 61/702,295, filed on Sep. 18, 2012.
(60) Provisional application No. 61/702,297, filed on Sep. 18, 2012.

(51) Int. Cl. A61K 31/69; C12Q 1/68; A61K 31/426; A61K 45/06

(52) U.S. Cl. CPC A61K 31/69; A61K 45/06; C12Q 1/6876; A61K 31/426
USPC 514/64; 544/229; 435/6.12; 548/200; 514/365; 562/7

(57) ABSTRACT

The present invention relates to compositions and methods for the treatment, prevention, and diagnosis of neuropathies due to PMP22 mis-expression in a subject having Charcot-Marie-Tooth disease, especially Charcot-Marie-Tooth 1A disease. The present invention incorporates the use of small molecule proteasome inhibitors such as, but not limited to, Bortezomib to inhibit or reduce the overexpression of the PMP2 gene.

### CROSS-REFERENCE

The present application claims the benefit of priority of U.S. Provisional Application No. 61/702,295, filed Sep. 18, 2012, and U.S. Provisional Application No. 61/702,297, filed Sep. 18, 2012, the disclosures of which are incorporated herein by reference.

### FIELD OF THE INVENTION

The present invention is directed to the specific use of pharmaceutical preparations of proteasome inhibitors, including but not limited to boronate derivatives, in the treatment of Charcot-Marie-Tooth disease, type 1A (CMT1A).

### BACKGROUND OF INVENTION

Charcot-Marie-Tooth (CMT) disease is the most commonly inherited peripheral neuropathy being found worldwide among all races and ethnic groups. Discovered in 1886 by three physicians, Jean-Martin Charcot, Pierre Marie, and Howard Henry Tooth, CMT affects an estimated 2.6 million people worldwide.

CMT is not usually life-threatening and almost never affects brain function. The disease is not contagious, but it is hereditary and can therefore be passed down from one generation to the next. CMT patients slowly lose normal use of their extremities as nerves degenerate and muscles weaken because the affected nerves no longer stimulate the muscles. Many patients also have some loss of sensory nerve functions.

The nervous system consists of motor neurons and sensory neurons. One set of nerves carries messages from the brain outward to the rest of the body and one brings messages from the extremities back to the brain. Messages that travel from the brain down the spinal cord, through the lower motor neurons (such as the sciatic nerve of the leg) to the muscles of the body are part of the motor neuron circuitry. Messages that travel upward from the sensory input to the spinal cord and finally the brain are sent by sensory neurons.

The peripheral nervous system is also comprised of motor and sensory nerve fibers, and since CMT affects the peripheral nerves, it results in both motor symptoms (weakness and muscle wasting) and sensory symptoms (numbness).

The peripheral nerves can be described as being somewhat like electrical wires with an inner core (the axon), which is wrapped in insulation (a sheath called myelin). When the myelin is damaged or compromised (as in Type 1 CMT), the nerve impulses are conducted more slowly than normal.

Type 1A is the most common form of CMT, comprising at least 60 per cent of all patients with Type 1 CMT. The disorder is caused by a duplication of the Peripheral Myelin Protein 22 (PMP22) gene on Chromosome 17. Instead of having two copies of the gene (one of each paired chromosome), there are three copies, two on one chromosome and one on the other. PMP22 is a peripheral myelin protein, but its exact function in causing CMT is still not known. It is inherited in an autosomal dominant fashion.

In a 1999 article by S. Niemann and co-authors (The “CMT rat”: peripheral neuropathy and dysmyelination caused by transgenic overexpression of PMP22. Ann. N.Y. Acad. Sci. 1999, 883 (Charcot-Marie-Tooth Disorders), 254-261), a transgenic rat model of Type 1A CMT (CMT1A) is described which provides formal evidence that this neuropathy can be caused by increased expression of PMP22.

CMT1A usually presents with a typical CMT phenotype (clinical presentation). Sufferers are slow runners in childhood, develop high arches, hammertoes and often require orthotics (braces) for ankle support. Varying degrees of hand weakness occur, often appearing as much as ten years after foot and leg problems. Problems with balance because of ankle weakness and loss of proprioception are common. Most patients remain ambulatory throughout life and life expectancy is normal.

Treatment of CMT is carried out in conjunction with medical professionals of various specialties. After diagnosis by a neurologist, CMT patients are usually directed to either a podiatrist for care of their foot problems, an orthotist for the manufacture and fitting of braces, an orthopaedic surgeon for surgeries to straighten toes, lengthen heel cords or lower arches, or a physical therapist or occupational therapist to design exercise programs to strengthen muscles or learn energy conservation.

At the present time there is no available pharmacological therapy that resolves CMT disorder. The International application PCT/FR2003/002236 (publication number WO2004/006911) relates to the use of a cAMP modulator in the preparation of compositions that are intended for the prevention or treatment of peripheral neuropathies including Type 1 CMT.

The present invention describes methods for the treatment of Charcot-Marie-Tooth disease comprising the administration to a subject affected by or presenting a risk of developing such disease, a composition consisting essentially of a therapeutically effective amount of at least one therapeutically active pharmaceutical composition of a proteasome inhibitor, or combination treatments comprising a proteasome inhibitor in combination with another pharmaceutically-active agent which is beneficial for the treatment of the symptoms of Type 1A Charcot-Marie-Tooth disorder.

### SUMMARY OF THE INVENTION

The present invention is directed to novel use of small-molecule proteasome inhibitors in the treatment of neuropathies due to PMP22 mis-expression in CMT1A.

Bortezomib (originally coded PS-341, and marketed as Velcade by Millennium Pharmaceuticals) is the approved name of the chemical entity [(1R)-3-methyl-1-({(2S)-3-phenyl-2-[(pyrazin-2-ylcarbonyl)amino]propanoyl}amino)butyl]boronic acid shown in FIG. 1. The drug is a therapeutically effective proteasome inhibitor approved in the U.S. for treating relapsed multiple myeloma and mantle cell lymphoma.

Additional examples of preferred proteasome inhibitors include, but are not limited to:
1) Ixazomib (MLN 2238); (R)-1-(2-(2,5-dichlorobenzamido)acetamido)-3-methylbutylboronic acid.
2) MLN 9708; 4-(carboxymethyl)-2-((R)-1-(2-(2,5-dichlorobenzamido)acetamido)-3-methylbutyl)-6-oxo-1,3,2-dioxaborinane-4-carboxylic acid.
3) Delanzomib (CEP-18770); [(1R)-1-[[(2S,3R)-3-hydroxy-2-[[(6-phenylpyridin-2-yl)carbonyl]amino]-1-oxobutyl]amino]-3-methylbutyl]boronic acid
4) Carfilzomib (PR-171); (S)-4-nethyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide
5) Oprozomib; (ONX-0912); O-methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide
6) YU 101; (αS)-α-(acetylamino)benzenebutanoyl-L-leucyl-N-[(1S)-3-methyl-1-[[(2R)-2-methyl-2-oxiranyl]carbonyl]butyl]-L-phenylalaninamide
7) Marizomib; (NPI-0052); (4R,5S)-4-(2-chloroethyl)-1-((1S)-cyclohex-2-enyl-(hydroxy)methyl)-5-methyl-6-oxa-2-azabicyclo[3.2.0]heptane-3,7-dione
8) Disufiram; [Disulfanediylbis(carbonothioylnitrilo)]tetraethane

Bortezomib has been shown to affect the development of neuropathies in patients undergoing treatment, indicating that proteasomal inhibition may be involved in the regulation of genes such as PMP22 that are intrinsic to the pathology of CMT1A. See, for example, the 2010 article by V. Csizmadia and co-authors (Effect of proteasome inhibitors with different chemical structures on the ubiquitin-proteasome system in vitro. Vet. Pathol. 2010, 47, 358-367).

In the present invention a novel cell-based gene reporter assay that faithfully replicates the CMT1A overexpression of PMP22 has been utilized to show that proteasome inhibitors such as Bortezomib can down regulate PMP22 expression in vitro. In some cases, when evaluation is in a Schwann cell line (S16), changes in native PMP22 expression in response to the effect of a proteasome inhibitor drug are measured by quantitative reverse transcriptase PCR (rtPCR).

In a further embodiment of this invention a rat model of human CMT1A has been employed to replicate these findings in vivo, in order to further demonstrate the utility of proteasome inhibitors such as Bortezomib, Ixazomib, Delanzomib, Carfilzomib, Marizomib or Disulfiram in the alteration of neurological function in these animals following treatment.

### DESCRIPTION OF FIGURES

FIG. 1: Chemical structures of Preferred Proteasome Inhibitors

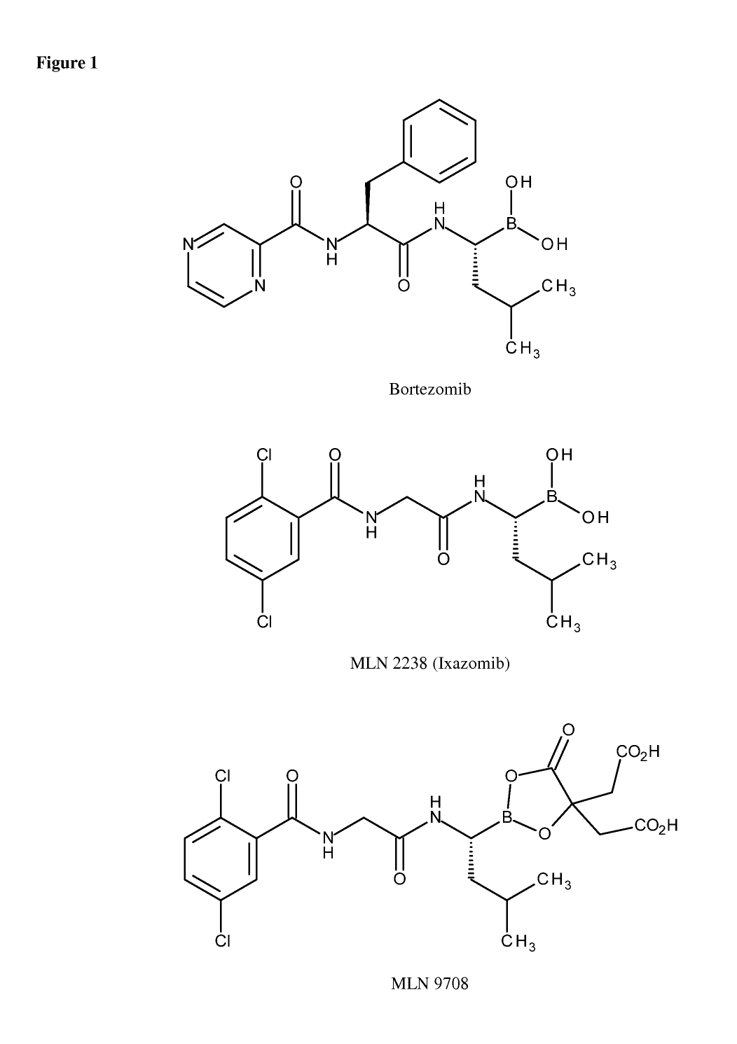

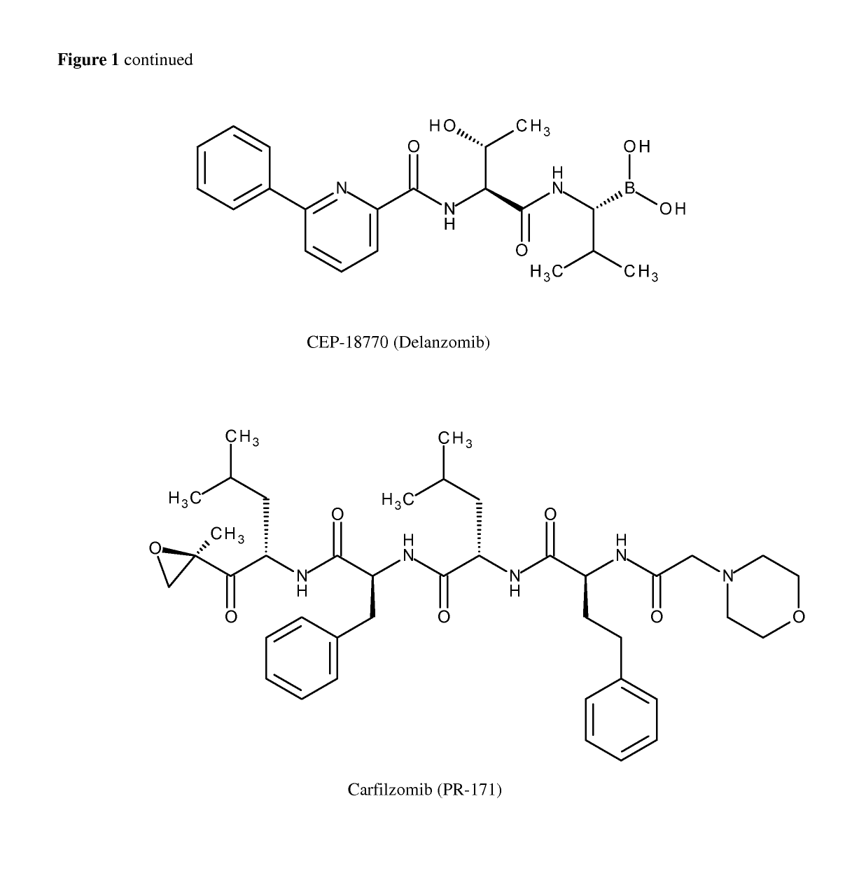

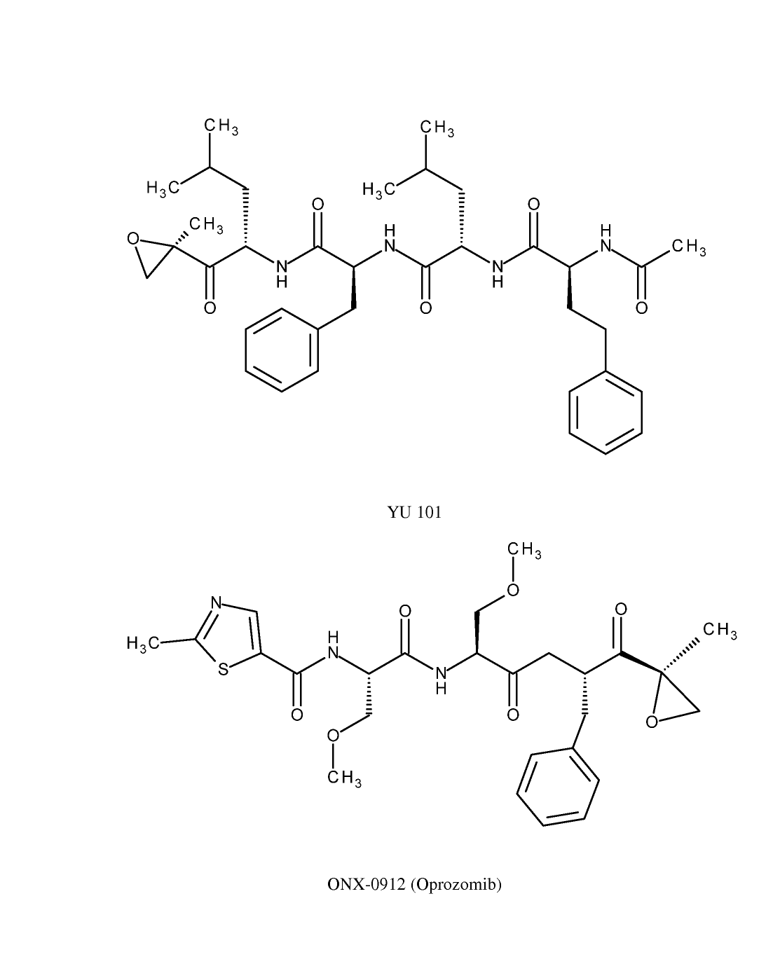

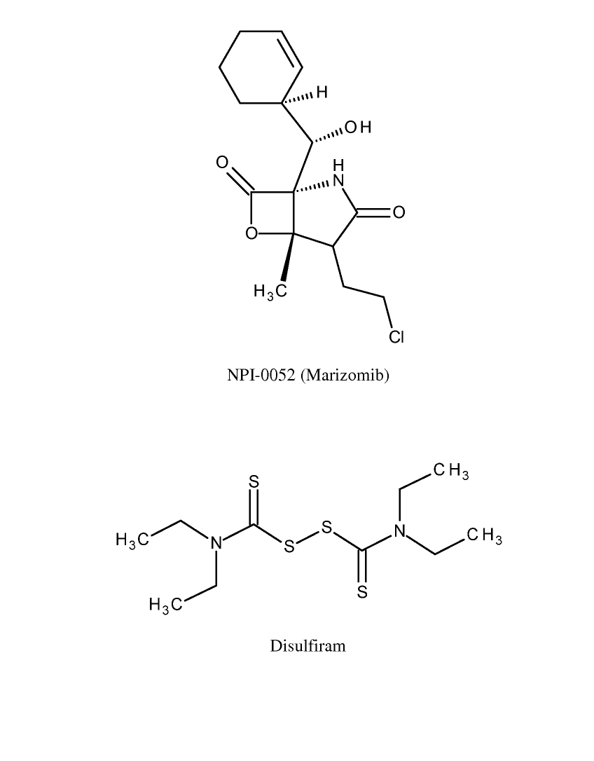

### DETAILED DESCRIPTION OF THE INVENTION

The present invention is directed to a novel method of use of proteasome inhibitors such as Bortezomib, as well as a series of second generation inhibitors. Second generation proteasome inhibitors have been reviewed recently by C. J. Kirk (Discovery and Development of Second-Generation Proteasome Inhibitors. Semin. Hematol. 2012, 49, 207-214) and by P. Lawasut and co-authors (New proteasome inhibitors in myeloma. Curr. Hematol. Malig. Rep. 2012, 7, 258-266).

This novel use is derived from evidence of a role in reducing PMP22 expression in vitro which in turn demonstrates utility in specific peripheral neuropathies such as Charcot Marie Tooth Disorder. The evidence of utility is further supported by a selective down regulation of

PMP22 gene expression in a rat model of CMT1A such as to be correlated with an improvement in measures of peripheral nerve function. The mechanism of drug action may involve proteasome inhibition, or other pharmacological activities which may be attributed to proteasome inhibitors. Some of the efficacious compounds of the present invention for treatment, be they used symptomatically or prophylactically for treatment of peripheral neuropathies such as Charcot Marie Tooth Disorder, may be known principally for alternate pharmacological activities, such as is the case for Disulfiram, but still be very effective proteasome inhibitors when dosed in humans; see, for example, the 2008 paper by B. Cvek and Z. Dvorak (The value of proteasome inhibition in cancer. Can the old drug, disulfiram, have a bright new future as a novel proteasome inhibitor? Drug Disc. Today, 2008, 13, 716-722).

A proteasome inhibitor such as Bortezomib can be administered to CMT1A patients by 3-5 second intravenous bolus (1 mg/ml) on days 1, 4, 8, and 11 of a 21 day cycle, for up to eight cycles.

Various documents including publications are recited throughout this disclosure. All such documents are hereby incorporated by reference. Trade names for the pharmaceutical composition are also noted.

The pharmaceutical composition for a representative boronate proteasome inhibitor, [(1R)-3-methyl-1-({(2S)-3-phenyl-2-[(pyrazin-2-ylcarbonyl)amino]propanoyl} amino)butyl]boronic acid, is referred to as Bortezomib, and marketed as Velcade™ [Millennium Pharmaceuticals, Inc.] is a 26S proteasome inhibitor that is approved for use in treating various neoplastic diseases, and especially treatment of relapsed multiple myeloma and mantle cell lymphoma. It is believed that the boron atom in bortezomib binds to the catalytic site of the proteasome, ultimately leading to proteasome inhibition and reduced degradation of pro-apoptotic factors, which in turn triggers apoptosis in treated cells. Bortezomib and related compounds are described in the following U.S. Pat. Nos.: 5,780,454, 6,083,903, 6,297,217, 6,617,317, 6,713,446, 6,747,150, 6,958,319, 7,119,080.

A cross-validating pair of orthogonal reporter assays, which employ either firefly luciferase or β-lactamase coupled to the intronic regulatory element of the human PMP22 gene, were used to measure CMT1A PMP22 overexpression as a means of identifying transcriptional inhibitors with therapeutic value toward CMT1A. During the targeted screening of a selected series of small molecules in these assays, proteasome inhibitors including Bortezomib were identified as selectively reducing CMT1A expression. A description of assays and methods used to identify compounds has been provided by S-W Jang and co-authors (Identification of drug modulators targeting gene-dosage disease. ACS Chem Biol., 2012, 7, 1205-1213).

The further ability of proteasome inhibitors such as Bortezomib to lower PMP22 expression in vivo was demonstrated by utilizing the rat model of human CMT1A described by S.M Sereda and co-authors (A transgenic rat model of Charcot-Marie-Tooth disease. Neuron, 1996, 16, 1049-1060). Test compounds were administered subcutaneously, intraperitoneally, or orally at doses which mimic a pre-clinically or clinically effective drug exposure but do not exceed the maximum tolerated dosage, as reported for MLN9708 by E. Kupperman and co-authors (Evaluation of the proteasome inhibitor MLN9708 in preclinical models of human cancer. Cancer Res., 2010, 70, 1970-1980). Abundance of gene expression could be determined relative to an untreated sample for each gene using the comparative Ct method as described in the procedure published by K. J. Livak and T. D. Schmittgen (Analysis of relative gene expression data using real-time quantitative PCR and the 2^{ΔΔC}T method. Methods, 2001, 25, 402-408).

Drug exposure following subcutaneous administration has been reported to be similar to that following intravenous administration, and is directly comparable, as reported by P. Moreau and co-authors (Prospective comparison of subcutaneous versus intravenous administration of bortezomib in patients with multiple myeloma. Haematologica. 2008, 93, 1908-1911). Changes in the expression of peripheral and central nervous system gene makers (including myelin markers such as PMP22, P0, and Myelin Basic Protein) were followed after proteasome inhibitor administration, and a reduction of PMP22 expression was observed. Changes in PMP22 gene expression were correlated with measures of neurological function that are used to measure the CMT1A neuropathy. These measures of neurological function included measures of motor coordination, nerve conduction velocity, and histology of femoral motor and sensory nerves. Relevant measures of CMT1A function have been outlined by J. C. Noreel and co-authors (Behavioural profiling of a murine Charcot-Marie-Tooth disease type 1A model. Eur. J. Neurosci. 2001, 13, 1625-1634).

Acid addition salts of the proteasome inhibitor and other agents employed in the invention can be prepared in a conventional manner by treating a solution or suspension of the corresponding free base with one chemical equivalent of a pharmaceutically acceptable acid.

Conventional concentration or crystallization techniques can be employed to isolate the salts. Illustrative of suitable acids are acetic, lactic, succinic, maleic, tartaric, citric, gluconic, ascorbic, benzoic, cinnamic, fumaric, sulfuric, phosphoric, hydrochloric, hydrobromic, hydroiodic, sulfamic, sulfonic acids such as methanesulfonic, benzene sulfonic, p-toluenesulfonic, and related organic or inorganic acids. The proteasome inhibitors and their pharmaceutically acceptable salts, may be administered alone or in combination with pharmaceutically acceptable carriers, in either single or multiple doses. Suitable pharmaceutical carriers include inert solid diluents or fillers, sterile aqueous solutions, oils (e.g. peanut oil, sesame oil) and various organic solvents. Enteric coated tablets may be a preferred formulation when the proteasome inhibitor utilized is orally active. The pharmaceutical compositions formed by combining the proteasome inhibitor and pharmaceutically acceptable carriers can be readily administered in a variety of dosage forms such as tablets, powders, lozenges, emulsions, oil soft gels, syrups, injectable solutions and the like. These pharmaceutical compositions can, if desired, contain additional ingredients such as flavorings, binders, excipients, taste-masking agents and the like. Thus, for purposes of oral administration, tablets containing various excipients such as sodium citrate, calcium carbonate and calcium phosphate may be employed along with various disintegrants such as starch, methylcellulose, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tableting purposes.

Solid compositions of a similar type may also be employed as fillers in soft and hard filled gelatin capsules. Preferred materials for this include lactose or milk sugar and high molecular weight polyethylene glycols. When aqueous suspensions, elixirs or beverages are desired for oral administration, the essential active ingredient therein may be combined with a large range of various sweetening or flavoring agents, coloring matter or dyes and, if desired, emulsifying or suspending agents, together with diluents such as water, ethanol, propylene glycol, glycerin and combinations thereof.

For parenteral administration, solutions containing the proteasome inhibitor or a pharmaceutically acceptable salt thereof in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media employed are all readily available by standard techniques known to those skilled in the art.

The effective dosages for the proteasome inhibitor employed in the methods of this invention will depend on the intended route of administration and factors such as the age and weight of the patient. The dosages will also depend on the particular form of the condition to be treated, whether symptomatically or prophylactically, especially in the case of onset and/or progression of symptoms associated with for example CMT1A. Doses will generally range from about 0.1 to about 300 mg/kg body weight of the patient per day, with administration carried out in single or divided dosages.

### Methods of Use

The methods of the present invention comprise administration (intravenous, subcutaneous or other suitable route of administration) of proteasome inhibitors such as Bortezomib, Ixazomib, Delanzomib, Carfilzomib, Marizomib, Disulfiram, or alternate clinically-effective proteasome inhibitors, to a mammal, preferably a human, to effect neurological function indicative of a CMT1A pathology.

The following are non-limiting examples of the present composition prepared utilizing conventional methods. The following example is provided to illustrate the invention and is not intended to limit the scope thereof in any manner.

### EXAMPLE 1

### PMP22 Expression in S16 Schwann Cells.

To determine if proteasome inhibitors affect endogenous PMP22 expression, quantitative rtPCR was utilized to measure the abundance of PMP22 mRNA transcript in the S16 Schwann cells, which were plated in 6 well plates and treated with each compound at the indicated concentrations. After 24 h., the cells were harvested to purify RNA, 1 μg of which was converted into cDNA. Quantitative rtPCR was performed in either SYBR green-based reactions or Taqman-based customized 384-well micro fluidic arrays using a ViiA7 system or StepOne Plus (Applied Biosystems). Abundance of gene expression was first normalized to beta-actin (ActB, NM_{—}031144.2) or 18 S rRNA, which served as an endogenous loading control across samples, and then determined relative to the untreated sample for each gene using the comparative Ct method described by K. J. Livak and T. D. Schmittgen (Analysis of relative gene expression data using real-time quantitative PCR and the 2^{ΔΔC}T method. Methods, 2001, 25, 402-408).

| Compound | Concentration | PMP22 P1 | PMP22 P2 |
|---|---|---|---|
| Control | | 1.011 ± 0.15 | 1.007 ± 0.12 |
| Carfilzomib | 1 nM | 0.003 ± 0.0004 | 0.012 ± 0.004 |
| Carfilzomib | 2.5 nM | 0.006 ± 0.0005 | 0.027 ± 0.0037 |
| Carfilzomib | 5 nM | 0.006 ± 0.0019 | 0.037 ± 0.00026 |
| carfilzomib | 10 nM | 0.014 ± 0.0007 | 0.046 ± 0.0022 |
| carfilzomib | 100 nM | 0.009 ± 0.0055 | 0.027 ± 0.017 |
| carfilzomib | 1 μM | 0.008 ± 0.0024 | 0.036 ± 0.0049 |
| MLN2238 | 25 nM | 1.177 ± 0.42 | 0.837 ± 0.041 |
| MLN2238 | 50 nM | 0.826 ± 0.225 | 0.865 ± 0.141 |
| MLN2238 | 100 nM | 0.005 ± 0.0005 | 0.020 ± 0.005 |
| MLN2238 | 1 μM | 0.003 ± 0.0007 | 0.022 ± 0.0026 |
| MLN2238 | 10 μM | 0.007 ± 0.00003 | 0.018 ± 0.0123 |

S16 Schwann cells were treated with indicated proteasome inhibitors for 24 h. RNA was prepared and analyzed by quantitative rtPCR using promoter specific primers for PMP22 (labeled P1 and P2). Relative levels of expression are shown relative to untreated control cells. Values are normalized to 18S rRNA. Errors reflect the standard deviation (S.D.) of technical duplicates.

| Sample Name | Target Name | RQ |
|---|---|---|
| 1_untreated | PMP22-P1 | 1.000 |
| 11_Borte_10 nM | PMP22-P1 | 0.517 |
| 12_Borte_100 nM | PMP22-P1 | 0.032 |
| 13_Borte_1 μM | PMP22-P1 | 0.041 |
| 14_Borte_10 μM | PMP22-P1 | 0.077 |
| 1_untreated | PMP22-P2 | 1.000 |
| 11_Borte_10 nM | PMP22-P2 | 0.809 |
| 12_Borte_100 nM | PMP22-P2 | 0.153 |
| 13_Borte_1 μM | PMP22-P2 | 0.232 |
| 14_Borte_10 μM | PMP22-P2 | 0.285 |

RQ=Relative Quantity, normalized to beta-actin

PMP22-1a:
GGCTCTCGATTGCAAAGAAATC, CAGGATCCCCAACAAGAGTAG
6FAM-AACTCCCAGCCACCATGCTTC-MGBNFQ
PMP22-1b:
GTTTGTGCCTGAGGCTACTC, CAGGATCCCCAACAAGAGTAG
6FAM-AACTCCCAGCCACCATGCTTC-MGBNFQ

### EXAMPLE 2

### Effect of MLN9708 on PMP22 Expression in Mice.

Results show relative levels of PMP22 after a single subcutaneous (s.c.) injection of MLN9708 at a dose equivalent to 10 mg substance/kg of body weight. MLN9708. Anatomical areas of subcutaneous administration could include the right or left thigh, or abdomen. The same anatomical area can be used for repeated administration. However, injections are typically to be rotated between sites (see; Fledrich, R. M. et al., Murine therapeutic models for Charcot-Marie-Tooth (CMT) disease. British Medical Bulletin, 2012, 102, 89-113).

| Compound | Concentration | Total PMP22 |
|---|---|---|
| MLN9708, n = 4 | 10 mg/kg | 1.00 ± 0.09 |
| Vehicle, n = 5 | | 0.739 ± 0.07 |

### EXAMPLE 3

Effect of Bortezomib on PMP22 expression in CMT1A rats.

Bortezomib was administered subcutaneously (s.c.) at a dose equivalent to 0.1 mg substance/kg of body weight, using an injection concentration of 2.5 mg of substance/ml. RNA was purified from sciatic nerve at 7 d post-injection. Results show relative levels of PMP22 and the 2 isoforms as determined by quantitative rtPCR. Errors indicate the standard error of the mean (SEM).

| Drug | Gene | Vehicle n = 8 | Treated n = 9 |
|---|---|---|---|
| Bortezomib 0.1 mg/kg | Total PMP22 | 1.00 ± 0.03 | 0.3 ± 0.05 |
| 7 d post-injection | PMP22 P1 | 1.00 ± 0.10 | 0.675 ± 0.17 |
| | PMP22 P2 | 1.00 ± 0.09 | 0.4 ± 0.07 |

### EXAMPLE 4

Effect of Oprozomib (ONX-0912) on PMP22 expression in CMT1A rats.

Oprozomib (ONX-0912) was administered at the indicated doses by oral gavage and RNA was purified from sciatic nerve at 7 d. Results show relative levels of PMP22 and the 2 isoforms as determined by quantitative rtPCR. Errors indicate SEM. Vehicle was 10% EtOH, 10% Tween80, 80% 10 mM citrate buffer pH=3.5 (see; Zhou, H-J. et al., Design and Synthesis of an Orally Bioavailable and Selective Peptide Epoxyketone Proteasome Inhibitor (PR-047). J. Med. Chem., 2009, 52, 3028-3038).

| Drug | Gene | Vehicle (n = 9) | Treated n = 7 |
|---|---|---|---|
| Oprozomib 10 mg/kg | Total PMP22 | 1.00 ± 0.07 | 0.62 ± 0.17 |
| | PMP22 P1 | 1.00 ± 0.16 | 0.46 ± 0.15 |
| | PMP22 P2 | 1.00 ± 0.09 | 3.1 ± 0.9 |
| Oprozomib 30 mg/kg | Total PMP22 | | 0.61 ± 0.07 |
| | PMP22 P1 | | 0.46 ± 0.1 |
| | PMP22 P2 | | 1.87 ± 0.77 |

Those skilled in the art will appreciate that numerous changes and modifications can be made to the embodiments of the invention and that such changes and modifications can be made without departing from the spirit of the invention. It is, therefore, intended that the appended claims cover all such equivalent variations as they fall within the true scope of the invention.

1. A method for treating peripheral neuropathy, said method comprising administering to a subject suspected of having peripheral neuropathy at least one proteasome inhibitor in a pharmaceutically acceptable carrier in an amount sufficient to reduce overexpression of PMP22.

2. The method of claim 1 wherein said peripheral neuropathy is CMT.

3. The method of claim 2 wherein CMT is CMT1A.

4. The method of claim 1 wherein at least one proteasome inhibitor is selected from a group consisting of Bortezomib (PS-341), Ixazomib (MLN 2238), MLN 9708, Delanzomib (CEP-18770), Carfilzomib (PR-171), YU101, Oprozomib (ONX-0912), Marizomib (NPI-0052) and Disufiram.

5. The method of claim 1 wherein the proteasome inhibitor is Bortezomib.

6. The method of claim 1 further having the addition of another pharmaceutically-active agent for the treatment of symptoms of said peripheral neuropathy.

7. The method of claim 6 wherein the addition of another pharmaceutically-active agent is a neuroprotective drug or an ion-channel blocking drug.

8. A method for preventing dysmyelination in a subject comprising administering at least one proteasome inhibitor in a pharmaceutically acceptable carrier an amount sufficient to prevent dysmyelination.

9. The method of claim 8 wherein at least one proteasome inhibitor is selected from a group consisting of Bortezomib (PS-341), Ixazomib (MLN 2238), MLN 9708, Delanzomib (CEP-18770), Carfilzomib (PR-171), YU101, Oprozomib (ONX-0912), Marizomib (NPI-0052) and Disufiram.

10. The method of claim 8 wherein the proteasome inhibitor is Bortezomib.

11. The method of claim 8 further having the addition of another pharmaceutically-active agent for the treatment of said peripheral neuropathy.

12. A pharmaceutical composition comprising an effective dose of at least one proteasome inhibitor for the treatment of CMTA disease.

13. A pharmaceutical composition of claim 12 having a pharmaceutically acceptable acid salt or a physiologically acceptable carrier.

14. A pharmaceutical composition of claim 12 wherein said acid salt is from a group consisting of acetic, lactic, succinic, maleic, tartaric, citric, gluconic, ascorbic, benzoic, cinnamic, fumaric, sulfuric, phosphoric, hydrochloric, hydrobromic, hydroiodic, sulfamic, sulfonic acids such as methanesulfonic, benzene sulfonic, p-toluenesulfonic, and related organic or inorganic acids

15. A pharmaceutical composition of claim 12 wherein said physiologically acceptable carrier is from a group consisting of inert solid diluents or fillers, sterile aqueous solutions, oils, and various organic solvents

16. A method for diagnosing peripheral neuropathy comprising:
a. obtaining a sample containing cells from a subject suspected of having peripheral neuropathy;
b. determining overexpression of PMP22 gene in the sample;
c. adding a proteasome inhibitor to reduce PMP22 gene overexpression; and
d. detection of a reduction in PMP22 gene overexpression wherein said detection is confirmation of peripheral neuropathy disease.

17. The method of claim 16 wherein said peripheral neuropathy is CMT.

18. The method of claim 17 wherein CMT is CMT1A.

19. The method of claim 16 wherein at least one proteasome inhibitor is selected from a group consisting of Bortezomib (PS-341), Ixazomib (MLN 2238), MLN 9708, Delanzomib (CEP-18770), Carfilzomib (PR-171), YU101, Oprozomib (ONX-0912), Marizomib (NPI-0052) and Disufiram.

20. The method of claim 16 wherein the proteasome inhibitor is Bortezomib.
